(19) United States
(12) Patent Application Publication
(10) Pub. No.: US 2003/0069551 A1
(43) Pub. Date: Apr. 10, 2003

(54) Multipurpose drain

(75) Inventors: Edward L. Bradley III, Sarasota, FL (US); Ridwan Hardy, Temple City, CA (US); J. Rae Walker, Rancho Dominguez, CA (US)

Correspondence Address: OSTROLENK FABER GERB & SOFFEN, 1180 AVENUE OF THE AMERICAS, NEW YORK, NY, 100368403

(73) Assignee: Axiom Medical, Inc.

(21) Appl. No.: 10/265,602

(22) Filed: Oct. 3, 2002

Related U.S. Application Data
(60) Provisional application No. 60/327,266, filed on Oct. 5, 2001.

(51) Int. Cl. A61M 1/00; A61M 27/00

(52) U.S. Cl. 604/276; 604/541

(57) ABSTRACT

A multipurpose drain has at least first and second lumens for selectively supplying medical material into a surgical site and removing the medical material and bodily fluids from the surgical site. The first and second lumens are extruded from suitable surgically compatible material to be detachably coupled to one another. The first and second lumens have a first open distal end and a second open distal end, respectively. The first and second distal ends are displaceable relative to one another to prevent the medical material from directly passing between the first and second open distal ends without first passing through the surgical site.

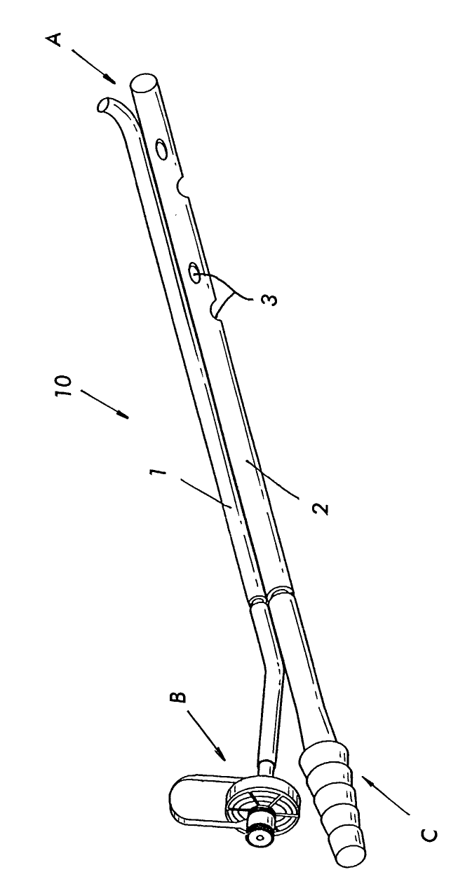

### CROSS REFERENCE TO RELATED APPLICATIONS

This application claims the benefit and priority of U.S. Provisional Application No. 60/327,266 filed Oct. 5, 2001 and entitled MULTIPURPOSE DRAIN, the disclosure of which is incorporated herein by reference.

### BACKGROUND OF THE INVENTION

The present invention relates to a multipurpose drain for medical irrigation and/or suction.

The following U.S. patents disclose surgical drains and related devices:

Des. 265,585
Des. 288,962
Des. 303,840
3,958,562
3,995,623
4,099,528
4,134,402
4,144,884
4,202,332
4,257,422
4,270,535
4,398,910
4,445,897
4,465,481
4,523,920
4,543,087
4,568,329
4,643,711
4,643,720
4,675,010
4,692,141
4,692,153
4,715,855
4,740,195
4,808,155
5,030,202
5,045,075
5,056,517
5,116,310
5,141,503
5,197,951
5,197,976
5,286,262
5,320,599
5,336,171
5,370,610
5,395,315
5,464,394
5,472,435
5,628,733
5,662,695
5,735,813
5,795,326
5,891,111
5,944,703
5,968,009
6,001,078
6,001,079
6,007,521
6,013,068
6,021,534
6,024,731
6,090,069
6,099,513
6,159,177
6,217,556 B1
6,299,593 B1
6,299,598 B1
6,313,190 B1
6,409,700 B1
6,443,156 B1

None of the known prior art, however, either discloses or suggests the invention herein.

### SUMMARY OF THE INVENTION

The disclosed multipurpose drain comprises a separable two-lumen catheter, one lumen preferably having a bacterial filter; the other lumen preferably having a suction connector and/or lateral drainage openings.

Other features and advantages of the present invention will become apparent from the following description of an embodiment of the invention which refers to the accompanying drawings.

### BRIEF DESCRIPTION OF THE DRAWING

The single FIGURE shows a two-lumen catheter according to an embodiment of the invention.

### DETAILED DESCRIPTION OF AN EMBODIMENT OF THE INVENTION

The FIGURE shows a two-lumen catheter 10 according to an embodiment of the invention. The lumens 1 and 2 and the overall catheter 10 may have any selected length and/or diameter according to surgical requirements.

According to a preferred manufacturing method, the two lumens may be extruded together from any suitable surgically compatible material, and are connected together by a weak portion of such material so that they are easily separable along their length.

Note that more than two lumens may be provided for the intended purpose. Furthermore, the lumens can be manufactured separately from one another and connected together by means of any suitable connector allowing the lumens to be oriented relative to one another to meet requirements imposed by any given medical procedure.

Preferably, at a distal end A, the first lumen 1 and the second lumen 2 are both open, and the second lumen 2 has additional openings 3 for providing drainage at a surgical site. These additional openings can have a uniform diameter or different diameters and can be arranged in any suitable pattern to control a rate at which drainage is conducted.

At a proximal end thereof, the second lumen 2 has a connector C which can be removably connected to a suction device for enhancing the drainage from the surgical site. Thus, the second lumen provides a channel for removing materials such as bodily fluids and exudates from a confined post-operative region.

The first lumen 1 has a filter B at the proximal end, for filtering a gas or liquid which may be supplied at that end to the first lumen 1. The filter may be removably mounted at the proximal end, or may be fixed to this end. A gas may be supplied through the filter for pressurizing the site in order to further enhance drainage. A liquid may be supplied through the filter for providing both enhanced drainage and irrigation at the surgical site.

Thus, the overall catheter can function in three modes: (a) simple drainage via the second lumen; (b) sump drainage, wherein a gas is introduced through the first lumen to enhance drainage through the second lumen; and (c) a closed irrigation-suction system, in which an irrigating liquid is supplied to the operative site through the first lumen, irrigates the site, and exits through the second lumen along with other materials to be removed. In the second and third modes, the introduced gas or liquid is filtered by the filter B to maintain sterile conditions.

The distal ends of the lumens 1 and 2 can be separated by any desired distance, by gently pulling them apart, in order to further enhance irrigation at the operative site. Advantageously, the distal ends are separated sufficiently to prevent a siphon effect in which irrigation fluid passes directly from the first lumen into the second lumen and thereby fails to provide the intended irrigation.

In addition to the irrigation and suction functions described above, the catheter 10 can be used for: (1) the introduction of various gases, fluids and other materials for use in treatment; (2) insertion of filtering or administration devices; and (3) introduction of a guiding device for positioning and placement of a smaller tube. All of these applications may be carried out using the first lumen, for example, at the same time the second lumen may be utilized for conducting drainage.

Although the present invention has been described in relation to a particular embodiment thereof, many other variations and modifications and other uses will become apparent to those skilled in the art. Therefore, the present invention is not limited by the specific disclosure herein.

What is claimed is:

1. A catheter comprising at least two lumens each having a respective distal end, the at least two lumens being operative to selectively supply a medical fluid material into a surgical site through the distal end of one of the at least two lumens and to drain the surgical site or a confined post-operative region through the distal end of the other lumen, the distal ends of the at least two lumens being displaceable relative to one another, the at least two lumens being comprised of a suitable surgically compatible material and being detachably coupled with one another.

2. The catheter defined in claim 1, wherein the distal ends of the at least two lumens are displaceable by a variable distance from each other, whereby the relative displacement of the distal ends of the at least two lumens is effective to prevent the medical material from passing directly between the distal ends of the one and other lumens without first irrigating the surgical site.

3. The catheter defined in claim 1, wherein each of the at least two lumens has a respective proximal end spaced from the distal end and being in flow communication therewith, wherein the proximal ends of the at least two lumens are open and displaceable relative to one another.

4. The catheter defined in claim 3, further comprising a filter coupled to the open proximal end of one of the at least two lumens and operative to filter the medical fluid material, said material being comprised of a gas or liquid for enhancing drainage of the surgical site or for enhancing irrigation thereof.

5. The catheter defined in claim 4, wherein the filter is detachably mounted to the proximal end of the one lumen.

6. The catheter defined in claim 4, wherein the one lumen is shaped and sized to receive administration or guiding devices removably insertable into the surgical site through the open proximal and distal ends of the one lumen.

7. The catheter defined in claim 4, further comprising a connector mounted to the proximal end of the other lumen to couple a suction and/or drainage device to said other lumen.

8. The catheter defined in claim 1, wherein the distal end of the other lumen has at least one lateral opening for providing enhanced drainage at the surgical site.

9. The catheter defined in claim 8, wherein the distal end of the other lumen is provided with at least two spaced lateral openings having a uniform diameter.

10. The catheter defined in claim 8, wherein the distal end of the other lumen is provided with at least two spaced lateral openings having different diameters.

11. A catheter comprising first and second lumens detachably coupled to one another for selectively supplying a gas or irrigating liquid into a surgical site and removing at least bodily fluids from the surgical site, wherein the catheter is operable in:
a simple drainage mode, in which only the bodily fluids are removed from the surgical site through the second lumen,
a sump drainage mode, in which the gas is introduced into the surgical site through the first lumen to enhance drainage of the bodily fluids through the second lumen, and
a closed irrigation mode, in which the irrigating liquid is supplied through the first lumen to irrigate the surgical site and is drained therefrom along with the bodily fluids through the second lumen.

12. The catheter defined in claim 11, wherein the first and second lumens are comprised of suitable surgically compatible material.

13. The catheter defined in claim 11, wherein each of the first and second lumens has respective distal and proximal open ends in fluid communication with one another, the distal ends of the first and second lumens being displaceable relative to one another to prevent the irrigating liquid from passing directly between the distal ends without first irrigating the surgical site.

14. The catheter defined in claim 13, wherein the distal end of the second lumen is provided with at least one lateral opening for removing the irrigating liquid and bodily fluids at the surgical site.

15. The catheter defined in claim 13, further comprising a filter coupled to the proximal open end of the first lumen and operative to filter the gas or irrigating liquid.

16. The catheter defined in claim 15, wherein the filter is detachably mounted to the proximal open end of the first lumen.

17. The catheter defined in claim 13, further comprising a connector mounted to the proximal end of the second lumen and operative to provide connection between a suction device and the second lumen for removing the irrigating liquid and bodily fluids from a confined post-operative region.

18. A multipurpose drain comprising first and second lumens co-extruded from suitable surgically compatible material so that the first and second lumens are detachably coupled to one another, the first and second lumens having a first distal end and a second distal end, respectively, and being operative to selectively supply medical material into a surgical site and removing the medical material and bodily fluids from the surgical site through the first and second distal ends, said first and second distal ends being separable relative to one another by a sufficient distance to prevent the medical material from directly passing between the first and second lumens without first passing through the surgical site.

19. A method for utilizing a multipurpose catheter having first and second lumens, the first and second lumens being connected to one another, the method comprising the steps of:
(a) introducing medical fluid into a surgical site through a distal end of the first lumen;
(b) removing the medical fluid and bodily fluids from the surgical site through a distal end of the second lumen; and
(c) during step (b), separating the distal ends of the first and second lumens from one another by a sufficient distance to reduce and/or eliminate a siphon effect between the first and second lumens in which the medical fluid passes directly from the one lumen into the other lumen without irrigating the surgical site.
